(19) United States
(12) Patent Application Publication
(10) Pub. No.: US 2013/0149428 A1
(43) Pub. Date: Jun. 13, 2013

(54) Fingerprint System

(71) Applicants: Phillip G. Pitts, Franklin, TN (US); James R. Vinson, Clarksville, TN (US)

(72) Inventors: Phillip G. Pitts, Franklin, TN (US); James R. Vinson, Clarksville, TN (US)

(73) Assignee: ENAMELITE, LLC, Clarksville, TN (US)

(21) Appl. No.: 13/758,987

(22) Filed: Feb. 4, 2013

Related U.S. Application Data
(63) Continuation-in-part of application No. PCT/US2011/046482, filed on Aug. 3, 2011.
(60) Provisional application No. 61/370,267, filed on Aug. 3, 2010.

(51) Int. Cl. A61B 5/117; B05B 1/00

(52) U.S. Cl. CPC A61B 5/1172; B05B 1/00
USPC 427/1; 239/589

(57) ABSTRACT

An aerosolized fingerprint compositions for spraying onto a surface for the purpose of indentifying latent fingerprints that comprises about 1 to about 75% fingerprint dusting powder and about 25 to about 99% non-CFC propellant. In certain embodiments an extender tube, optionally comprising an expander, is coupled to a aerosolized container and is in fluid communication with the aerosolized fingerprint composition.

### CROSS-REFERENCE TO RELATED APPLICATIONS

This application and is a continuation-in-part of PCT International Application No. PCT/US11/46482, filed Aug. 3, 2011, which claims the benefit of U.S. Provisional Application No. 61/370,267, filed Aug. 3, 2010, the contents of which are incorporated herein in their entirety by this reference.

### FIELD OF THE INVENTION

The present invention relates generally to the fingerprinting art by aiding a person in identifying and “lifting” fingerprints from a surface.

The present invention further relates to an aerosolized fingerprint formulation for aiding a person in identifying and “lifting” fingerprints from a surface.

Accordingly, the spray and method of the present invention increases the efficiency of obtaining a fingerprint while decreasing the amount of time and amount of materials usually required by a forensic technician to apply such coatings. The spray of the present invention provides an even coating and consistent quality application.

Additionally, the present invention is advantageous in that clean up is easier and allows for a greater variety of surfaces and angles to be dusted for prints.

### BACKGROUND OF THE INVENTION

Generally, a fingerprint is an impression of the friction ridges of all or any part of the finger. As used herein, “fingerprint” or “print” are used interchangeably, and refer to an impression of a friction ridge. Thus, use of the terms “fingerprint”, or “print” is intended to cover all traditional fingerprints, palm prints, toe prints, etc. A friction ridge is a raised portion of the epidermis on the palmar (palm), digits (fingers and toes), or plantar (sole) skin, consisting of one or more connected ridge units of friction ridge skin. These are sometimes known as “epidermal ridges” which are caused by the underlying interface between the dermal papillae of the dermis and the interpapillary (rete) pegs of the epidermis. These epidermal ridges serve to amplify vibrations triggered when fingertips brush across an uneven surface, better transmitting the signals to sensory nerves involved in fine texture perception. The ridges assist in gripping rough surfaces, as well as smooth wet surfaces.

Fingerprints may be deposited in natural secretions from the eccrine glands present in friction ridge skin (secretions consisting primarily of water) or they may be made by ink or contaminants transferred from the peaks of friction skin ridges to a relatively smooth surface such as a fingerprint card. The term fingerprint normally refers to impressions transferred from the pad on the last joint of fingers and thumbs, though fingerprint cards also typically record portions of lower joint areas of the fingers (which are also used to make identifications).

Fingerprint identification or palm print identification is the process of comparing questioned and known friction skin ridge impressions from fingers or palms or even toes to determine if the impressions are from the same finger or palm. The flexibility of friction ridge skin means that no two finger or palm prints are ever exactly alike (never identical in every detail), even two impressions recorded immediately after each other. Fingerprint identification (also referred to as individualization) occurs when an expert (or an expert computer system operating under threshold scoring rules) determines that two friction ridge impressions originated from the same finger or palm (or toe, sole) to the exclusion of all others.

A known print is the intentional recording of the friction ridges, usually with black printers ink rolled across a contrasting white background, typically a white card. Friction ridges can also be recorded digitally using a technique called Live-Scan. A latent print is the chance reproduction of the friction ridges deposited on the surface of an item. Latent prints are often fragmentary and may require chemical methods, powder, or alternative light sources in order to be visualized.

When friction ridges come in contact with a surface that is receptive to a print, material on the ridges, such as perspiration, oil, grease, ink, etc. can be transferred to the item. The factors which affect friction ridge impressions are numerous, thereby requiring examiners to undergo extensive and objective study in order to be trained to competency. Pliability of the skin, deposition pressure, slippage, the matrix, the surface, and the development medium are just some of the various factors which can cause a latent print to appear differently from the known recording of the same friction ridges. Indeed, the conditions of friction ridge deposition are unique and never duplicated.

There are several types of fingerprint types, including latent prints, patent prints, and plastic prints. For latent prints, although the word latent means hidden or invisible, in modern usage for forensic science the term latent prints means any chance of accidental impression left by friction ridge skin on a surface, regardless of whether it is visible or invisible at the time of deposition. Electronic, chemical and physical processing techniques permit visualization of invisible latent print residue whether they are from natural secretions of the eccrine glands present on friction ridge skin (which produce palmar sweat, consisting primarily of water with various salts and organic compounds in solution), or whether the impression is in a contaminant such as motor oil, blood, paint, ink, etc. There are different types of fingerprint patterns such as an arch, tented arch, a loop, and a whorl. Each indicate what type of fingerprint it is.

Latent prints may exhibit only a small portion of the surface of the finger and may be smudged, distorted, overlapping, or any combination, depending on how they were deposited. For these reasons, latent prints are an “inevitable source of error in making comparisons,” as they generally “contain less clarity, less content, and less undistorted information than a fingerprint taken under controlled conditions, and much, much less detail compared to the actual patterns of ridges and grooves of a finger.”

Patent prints are friction ridge impressions of unknown origins which are obvious to the human eye and are caused by a transfer of foreign material on the finger, onto a surface. Because they are already visible they need no enhancement, and are generally photographed instead of being lifted in the same manner as latent prints. An attempt to preserve the actual print is always made with numerous techniques; for later presentation in court. Finger deposits can include materials such as ink, dirt, or blood onto a surface.

A plastic print is a friction ridge impression from a finger or palm (or toe/foot) deposited in a material that retains the shape of the ridge detail. Commonly encountered examples are melted candle wax, putty removed from the perimeter of window panes and thick grease deposits on car parts. Such prints are already visible and need no enhancement, but investigators must not overlook the potential that invisible latent prints deposited by accomplices may also be on such surfaces. After photographically recording such prints, attempts should be made to develop other non-plastic impressions deposited at natural finger/palm secretions (eccrine gland secretions) or contaminates.

Since the late nineteenth century, fingerprint identification methods have been used by police agencies around the world to identify both suspected criminals as well as the victims of crime. The basis of the traditional fingerprinting technique is simple. The skin on the palmar surface of the hands and feet forms ridges, so-called papillary ridges, in patterns that are unique to each individual and which do not change over time. Even identical twins (who share their DNA) do not have identical fingerprints. Fingerprints on surfaces may be described as patent or latent. Patent fingerprints are left when a substance (such as paint, oil or blood) is transferred from the finger to a surface and are easily photographed without further processing. Latent fingerprints, in contrast, occur when the natural secretions of the skin are deposited on a surface through fingertip contact, and are usually not readily visible. The best way to render latent fingerprints visible, so that they can be photographed, is complex and depends, for example, on the type of surface involved. It is generally necessary to use a ‘developer’, usually a powder or chemical reagent, to produce a high degree of visual contrast between the ridge patterns and the surface on which the fingerprint was left.

Developing agents depend on the presence of organic materials or inorganic salts for their effectiveness although the water deposited may also take a key role. Fingerprints are typically formed from the aqueous based secretions of the eccrine glands of the fingers and palms with additional material from sebaceous glands primarily from the forehead. The latter contamination results from the common human behaviors of touching the face and hair.

The resulting latent fingerprints consist usually of a substantial proportion of water with small traces of amino acids, chlorides, etc., mixed with a fatty, sebaceous component which contains a number of fatty acids, triglycerides, etc. Detection of the small proportion of reactive organic material such as urea and amino acids is far from easy.

Crime scene fingerprints have typically been detected by simple powders, or some chemicals applied at the crime scene. The application of powders for the development of latent fingerprints is one of the earliest known techniques dating back to the nineteenth century. See Advances in Fingerprint Technology, Second Edition, Lee, H. and Gaensslen, R., Eds.; Forensic and Police Science Series; CRC Press: Boca Raton, 2001. The constituents of latent prints facilitate an adherence of powder particles thereby rendering impressions visible. Developed impressions may then be preserved by lifting or photography.

Over the years a number of different methods of powder application have been proposed. The most common method of application for conventional powder dusting is by brushing the surface with appropriate bristled brushes such as animal hair or fiberglass. A major drawback with the typical method is how the powders are packaged and applied. The powders are typically packaged in a jar. The fingerprint brush is dipped into the jar as to allow the powders to accumulate on the brush. They are later transferred from the brush to the surface. This method leads to inaccuracies in actualizing the print and is cumbersome.

Other, less popular, methods have been proposed such as atomizers, sifting, and aerosolized spray, but the results were typically inferior to the standard method of dusting using brushes. See Bridges, B. Practical Fingerprinting. Funk & Wagnalls Company: New York, 1963. Olsen, R. Scott's Fingerprint Mechanics. Charles C Thomas Publisher: Springfield, 1978. Chapel, C. Fingerprinting: A Manual of Identification. Coward McCann, Inc.: New York, 1941.

The atomizer method of applying fingerprint powders consists of blowing the fingerprint powder onto the surface by a blast of air thus reducing the need for physical contact that might destroy or damage the ridge detail. The blast of air from the old style atomizer charged with powder was not in itself strong enough to fully develop an impression and still required brushing to enhance the print. Atomizers had a tendency to paint the surface as the air forced the powder into the surface depressions and never delivered a very even spread of powder. This problem is similar to the previous one in that it is difficult to accurately actualize the print.

The sifting method of applying fingerprint powders consists of applying the powder directly onto the object to be processed and sliding the powder back and forth across the suspected area until enough powder adhered to the latent impressions. A light touchup is still necessary using a fingerprint brush to remove excess powder. This method often resulted in too much powder being applied to the surface thereby destroying or over powdering the print.

Aerosol powders have been tested in the past with inferior results to that of brushing, primarily due to improper air control, clogging of the nozzles causing uneven spray distribution, and poor powder ratios or propellants.

The present invention has many advantages over prior art methods.

### SUMMARY OF THE INVENTION

It is accordingly one object of the present invention to provide a spray delivery system, including an aerosolized delivery system that will overcome current limitations and disadvantages of lifting fingerprints.

Another object of the present invention is to provide a simple and dependable product and method for efficient and fast developing of latent prints.

Another object of the present invention is to provide a method of lifting prints that is versatile in terms if surfaces, objects, angles, etc. For example, embodiments of the present invention may lift prints from downwardly facing overhangs or surfaces.

Another object of the present invention is to provide a spray fingerprint composition that can be used in the process of identifying fingerprints on a surface.

Another object of the present invention is a method of obtaining fingerprints using a spray composition of the present invention.

Another embodiment of the present invention is an aerosolized spray fingerprinting formulation, comprising (weight %): about 1 to about 75% fingerprinting powder; and about 25 to about 99% non-CFC propellant. In some embodiments the fingerprint formulation contains no substance that is liquid when the formulation is in the container, when the formulation is sprayed, and/or when the formulation is applied onto a surface.

Another embodiment of the present invention comprises an extender tube in fluid communication with an aerosolized spray container, which may include a composition with a ratio of particles to propellant of about 1:30 to about 1:50, or preferably about 1:35 to about 1:45, or more preferably of about 1:40.

Another embodiment of the present invention is a method of obtaining fingerprints, comprising: (1) providing an aerosolized spray fingerprint dusting powder of the present invention; (2) identifying a surface that may contain a latent fingerprint; (3) spraying the surface with the fingerprint dusting powder to actualize latent print; and (4) analyzing actualized print.

Another embodiment of the present invention is method of obtaining fingerprints, comprising: providing an aerosolized container containing a composition including at least one fingerprint powder and a dry propellant, coupling an extender tube to the container, identifying a surface that may contain a latent fingerprint, aiming the extender tube at the surface, and spraying the composition through the extender tube onto the surface.

Another embodiment of the present invention comprises an extender tube. The extender tube may comprise an expander, optionally located at one end of the extender tube, to disperse a sprayed composition. The extender tube may be coupled to the container such that it is in fluid communication with the composition in the container. The extender tube may be separable or integral with the container. In certain embodiments, the extender tube may be coupled to the container via a valve stem that extends outwardly from the container.

Another embodiment of the present invention comprises a actuator. The actuator may be coupled to a container, optionally via a valve stem connected to the container. The actuator may be coupled to one end of the extender tube. The actuator may be permanently attached to either or both of the extender tube and the container. In certain embodiments, the actuator is coupled to both the container and the extender tube, and is configured such that the composition in the container may be sprayed out of the container, through the actuator and the extender tube, and out into the atmosphere and/or onto a surface.

Another embodiment of the present invention is a kit comprising a container containing a composition, wherein the composition includes a fingerprint powder and a dry propellant, and an extender tube, wherein the extender tube includes a first end and a second end, and wherein the first end is coupled to the container such that the extender tube is in fluid communication with the composition. The extender tube may be detachable or permanently coupled to the container.

Certain embodiments of kits comprising further items such as a second container containing the composition, which may be the same as or different from the composition in the first container, gloves, an ultraviolet light, a flashlight, a camera, lifting tape, and a brush. These items may be of any kind or design known in the art for identifying fingerprints.

Additionally, embodiments of the present invention utilize a dry powder aerosol. Unsuccessful prior art attempts contain a type of binding agent the artificially enhances the ability of the powders to bind to or stick to the sprayed surface. These methods are deficient in that they do not allow the powders to be easily worked to properly expose or develop the finger print because of the nature of the powder. The methods of the present invention comprise a dry powder system/dry propellant system that eliminates the attachment of a sticky layer of powder on a surface which would inhibit properly controlling a sprayed powder layer to expose a finger print.

Fingerprints are used to identify an unknown victim, witness, or suspect, to verify records, and most importantly, as links and matches between a suspect and a crime. Occasionally, a print is found that is made with the palm of the hand or a bare foot. These are ordinarily processed by the same methods used for fingerprints. Accordingly, as indicated herein, the term “fingerprint” is generic and is not strictly limited to fingers. It generically includes finger prints, palm prints, toe prints foot prints, and partial prints thereof.

Ridges develop on the skin of its fingers and thumbs. These ridges arrange themselves in more or less regular patterns. For purposes of classification, experts divide these ridge patterns into three basic classes: arches, loops, and whorls. When prints are found, an expert compares them with samples.

There are three basic forms of prints: plastic, which are impressions left in soft material like wax, paint, or putty; visible, which are made by blood, dirt, ink, or grease; and latent, which are normally invisible and must be developed before they can be seen and photographed.

The most common way of developing latent prints is by dusting with fingerprint powders. A very fine powder is gently brushed over the surface of an object suspected of having fingerprints. The fine powder sticks to the oils and perspiration that are left behind from the top of the friction ridges of the skin. Great care and skill are required to actualize the latent print. A non-skilled person may cause damage to the ridged line of the fingerprint during the brushing step.

One embodiment of the present invention is an aerosolized fingerprint powder composition, comprising (by weight %): about 1 to about 75% fingerprint dusting powder; and about 25 to about 99% non-CFC dry propellant. In some embodiments the formulation does not comprise any liquid substances.

Another embodiment of the present invention is a method of obtaining fingerprints, comprising: (1) providing an aerosolized spray fingerprint powder composition, comprising a fingerprint powder and a dry propellant; (2) providing an isolation device that comprises a small opening defined by at least one wall and a large opening defined by at least one wall; (3) identifying a surface that may contain a latent fingerprint; (4) placing the large opening of the isolation device over the surface; (5) spraying the aerosolized fingerprint powder into the small opening of the isolation device to actualize latent print; and (6) analyzing actualized print.

Another embodiment of the present invention is a kit, comprising: at least one aerosolized container that comprises a composition that includes a fingerprint formulation and a propellant; a finger print brush; an isolation device that is defined by at least one wall and has a fingerprint surface opening and a spray opening.

In any of the embodiments described herein, the container, and optionally the extender tube and/or isolation device, may be designed to be inconspicuous. For example, the container, and optionally the extender tube and/or isolation device as well, may be designed to look like a pen, camera, or other inconspicuous device.

As indicated herein, aerosol fingerprint powders have been tested in the past with inferior results to that of brushing. Reasons for this inferiority include improper air control, clogging of the nozzles causing uneven spray distribution, and poor powder ratios or propellants. See Olsen, R. Scott's Fingerprint Mechanics. Charles C Thomas Publisher: Springfield, 1978.

Prior art aerosol fingerprint methods did not utilize a portable “isolation device,” which maximizes the effectiveness of the aerosol spray delivery system by capturing the sprayed materials for maximum utilization. Additionally, prior aerosol fingerprint apparatuses and method did not utilize an “extender tube,” which as discussed herein, also provides numerous advantages.

Additionally, with respect to embodiments of the present invention, the powder to propellant ratio of the present invention is greater. Specific examples of the present invention include a formula that utilizes about 1 to 10 ratio (powder to propellant).

Certain embodiments of the present invention comprise: an aerosolized container with a composition that includes a fingerprint formulation and a propellant, wherein the aerosolized container further comprises an extender tube. Embodiments of extender tubes include an expander coupled to, for example, the end of the extender tube not coupled to the aerosolized container. Expanders are designed to, among other things, disperse the aerosolized spray as desired. Specific embodiments of the present invention include a composition having a ratio of about 1:30 to 1:40 (fingerprint powder to propellant).

Embodiments comprising extender tubes may require less material to identify a specific print, may be capable of analyzing smaller areas, and may be able to target more difficult to reach surfaces than designs without extender tubes and other prior designs.

In practice, the methods of the present invention actualize a print using a low powder concentration. The “low” powder concentration used in embodiments of the present invention unveils a completely new fingerprint revealing processes. Methods use such a small amount of powder with a single burst (less than prior methods), that the powder is basically “layered-on” the print as never before. For example, when using the isolation device and/or extender tube of the present invention, and using only 1 spray burst in the top of the isolation device and/or extender tube of a black finger print powder, a white substrate, the detection of the black powder not attached to the print with the naked eye is difficult. The method of the present invention typically leaves no “clumps” of powders on the sprayed surface.

To further explain the “layered” technique, with all past and current finger print powder application techniques (by brush, air, aerosol) all apply much more powder than necessary to expose and develop the print. All powder application techniques to date battle inefficiency. Simply stated, with all other know latent powder application techniques, the processes employed requires one to work or move the excess powders off or away from an exposed print, as not to damage or contaminate the print with too much powder. The method of the present invention is the first finger print powder delivery system ever which allows a minimal amount of powders to be applied (maybe less than is required to 100% develop the print) at one single application. With the method of the present invention, if the print is not exposed with the first applied powder amount (normally with a single spray burst) then an additional spray burst may be used to further highlight the print without the fear of “overexposing” the print with too much powder. The method of the present invention is designed to empower the user with a metered approach to powder application. The method of the present invention is highly safe, efficient and predictable for the user who might be a novice. Other powder techniques requires the user to move powders away from the surface, while the method of the present invention allows powders to be safely built up or layered on a surface. This is a much more effective and accurate fingerprint lifting system.

These and other objects will be apparent from the present disclosure and claims. Additional substance, advantages and other novel features of the invention will be set forth in part in the description that follows and in part will become apparent to those skilled in the art upon inspection of the following or may be learned with practice of the invention or improved development. Further, the above embodiments are examples of the present invention and not intended to be limiting thereof.

### BRIEF DESCRIPTION OF THE DRAWINGS

FIG. 1 is a drawing that shows an embodiment of a fingerprint formulation being sprayed into an isolation device of the present invention, over a surface that is suspected of having a latent print.

FIG. 2 is drawing that shows the same view as FIG. 1, but also providing a cut-away of the isolation device.

FIG. 3 is a side, cut-away view of FIG. 1.

FIG. 4 are photographs taken with a polarized light microscope at 200 times magnification, which show (A) commercial black fingerprint powder, and (B) black power applied in an method in accordance with an embodiment of the present invention.

FIG. 5 is a representation of powder from an embodiment of the present invention applied under a stereomicroscope at approximately 60 times magnification.

FIG. 6 is a drawing that shows a cut-away view indicating an embodiment of the present invention comprising an extender tube dispersing the particles onto the surface.

FIG. 7 is a drawing of the expander attached to the extender tube shown in FIG. 6.

FIG. 8 is a drawing that shows a cross-sectional view of the expander shown in FIG. 6.

FIG. 9 is a drawing that shows an internal view of the expander shown in FIG. 6 from an inlet side of the expander.

FIG. 10 is a drawing that shows the internal surface of an exit cap of the expander shown in FIG. 6.

### DETAILED DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a projection” includes a plurality of such projections, and so forth.

As stated above, embodiments of the present invention include a spray fingerprint composition that can be used in the process of locating and lifting fingerprints on a surface and a method of obtaining fingerprints from a surface.

The aerosolized spray fingerprint dusting powder compositions of the present invention may be used for obtaining fingerprints. This method comprises identifying a surface that may contain a latent fingerprint; spraying the surface with the fingerprint dusting powder to actualize latent print; and analyzing actualized print. Additionally, the actualized print may be documented by photographing the actualized print or removing the actualized print with an adhesive material.

Glass Frit

One optional component of the present invention is glass frit particles. Without being bound by theory or mechanism, glass frit, when used, may impart enhanced finger print ridge detail and clarity. Glass frit usable in connection with the present invention is available from many different manufacturers worldwide. In embodiments of the present invention, the same glass frit currently used in the dental profession and hobby ceramist practicing the brush technique may be used in the formulation.

The glass frit may be in the form of either a natural or man-made mixture of inorganic chemical substances. The glass frit is produced by rapidly quenching a molten, complex amalgamation of materials. Such glass frit is available from a number of manufacturers under varying designations.

In embodiments, at least about 90% of the frit have a particle size of about 25 microns and under. In another embodiment, at least about 90% of the frit have a particle size of about 20 microns and under. In another embodiment, at least about 75% of the frit have a particle size of about 15 microns and under. In another embodiment, at least about 90% of the frit have a particle size of about 15 microns and under. In another embodiment, at least about 75% of the frit have a particle size of about 10 microns and under. In another embodiment, at least about 90% of the frit have a particle size of about 10 microns and under. Further, in embodiments of the present invention, at least about 75% of the frit have a particle size of about 8 microns and under. In another embodiment, at least about 90% of the frit have a particle size of about 8 microns and under.

One source of glass frit is the Ferro Corporation, Coating Division, located in Cleveland, Ohio. 3227 leadless frit available from Ferro is an example of glass frit of the present invention. Further, the frit can be milled such as ball milled or jet milled in a ceramic mill to arrive at the preferred particle size.

Because of varying percentages of chemical components in the various glass frit formulations available from manufacturers, the intrinsic characteristics of the glass frit also vary. As a result, the particular weight percentages of the individual components of the glaze composition may need to be adjusted across the range set forth above to provide proper and consistent results.

In examples of the present invention, the glass frit is present in amounts ranging from about 4 to about 50 weight % of the total composition. In other examples, this glass frit is present in an amount of from about 8 to about 35%. In other examples, this range is from about 9 to about 17%.

Propellant

Prior art compositions for aerosol applications typically used, as a suspension agent, chlorofluorocarbons, which are now known to be hazardous. Examples include 1,1,1-trichloroethane and Freon TF 22 propellants. Inhalation or swallowing vapors may irritate the respiratory tract and affect the central nervous system. Over exposure symptoms include headache, dizziness, weakness, and nausea. Higher levels of exposure (>5000 ppm) can cause irregular heartbeat, liver, and kidney damage, fall in blood pressure, cardiovascular damage, unconsciousness and even death. 1,1,1-trichloroethane is also thought by some to be a possible carcinogen. Furthermore, CFC materials are the source of a myriad of environmental problems, including adversely affecting the ozone layer. Therefore, their use is not unacceptable in the fingerprinting formulations of the present invention. Accordingly, one advantage of the present invention is the non-CFC propellant.

The propellant used in connection with the present invention is a dry propellant. A dry propellant contains no additives that are added to bind or artificially adhere sprayed powder materials to each other or to a surface. The dry propellants of the present invention are hydrocarbon-based or compressed gas materials such as CO₂, propane, butane or any derivative thereof, or combinations thereof. It will be apparent to those of skill in the art that any non-CFC propellant that achieves desired results may be implemented in embodiments of the present invention, including both flammable and non-flammable propellants, both hydrocarbon and non-hydrocarbon propellants, and so forth.

The propellant used in connection with the present invention may also be a non-CFC propellant. One propellant that may be used is a hydrocarbon propellant. Further examples include isobutane, butane, or any mixtures thereof. The butane and isobutane hydrocarbon propellants are available, for example, from Aeropres Corporation, Shreveport, Louisiana under the designation of A-17, A-31 up to the strongest pressure of A-132 propellants

Certain embodiments comprise spray pressures that may range between about 17-132 psig. In some embodiments the best results and the most consistent spray characteristics may be obtained if the compositions of the present invention are packaged at a pressure in the range of between about 17-56 psig.

Propellants used in connection with certain embodiments of the present invention may comprise any non-flammable propellant that is suitable for particular applications. For example, non-flammable propellants may include hydrofluorocarbon propellants. Examples of hydrofluorocarbon propellants include 1,1,1,2-tetrafluoroethane, 1,1,1,3,3,3-hexafluoropropane, and 1,1,1,2,3,3,3-heptafluoropropane, which, for example, may be obtained from Dupont, located in Wilmington, Del., under the respective designations Dymel 134a/P, Dymel 236fa, and Dymel 227 ea/P.

The non-CFC propellant of the present invention may be present in the composition in amounts ranging anywhere from about 10-99%, about 50-99%, about 70-96%, about 80-93%, and/or about 85-92% by weight of the total composition.

Fingerprint Powder and Formulation

The fingerprint powder of the present invention is not known to be critical. That is, any fingerprint powder known in the art is capable of being used with the present invention. For example, the fingerprinting powder may be selected from the group consisting of non-metallic and non-fluorescent powders commonly used that can be colored black, white, silver, grey, red, etc., or combinations thereof. Fluorescent powders commonly used care colored red, green, yellow, orange, and blue. Metallic powders commonly used are colored silver, copper, red, orange, green, blue, yellow, etc., or combinations thereof. In embodiments, some types of latent print powders are comprised of a combination of materials to maximize the detection of latent fingerprints.

Powders of varying color are used to get the maximum contrast with the background material. The excess powder is blown off, leaving a clear impression from the powder that adheres to the ridges of the print. The print can then be photographed and lifted with an adhesive material such as tape.

The powders of the present invention may be comprises of various materials, including, for example, talc, silica, barium sulfate, calcium carbonate, gypsum, alumina, agalmatolite, lithopone, zinc oxide, silicon oxide, titanium oxide, carbon black, graphite, molybdenum disulfide, iron oxide, silica black, chrome black, mineral black, vine black, bone black, silicon carbonate, and mixtures thereof.

Certain embodiments of the present invention comprise a composition that includes two or more types of powders. The two or more powders may be selected from the same or different powder families or categories. One of ordinary skill in the art may select any number of powders, varying in type, color, material, and the like, and combine them in any suitable proportion to meet the limitations of a particular circumstance and/or increase the composition's functionality.

For example, in certain embodiments of the present invention a composition comprises both a black powder and an orange fluorescent powder. Such an embodiment allows a user to capture latent prints through two distinct processes. The traditional black powder may capture prints by making the print visible and detectable. The orange, or any other color, fluorescent powder is UV light activated, allowing for prints to be covertly discovered. Combined formulations may have the capability to detect finger prints in multiple different circumstances. Combined formulations may make embodiments easier to use and implement as well as reduce the number of spray varieties a user needs access to.

The following is a non-limiting list of powders that can be used in connection with the present invention. Like all examples presented herein, it is presented for exemplary purposes, and is not to be construed as being limiting of the present invention.

| | | | |
|---|---|---|---|
| Powder Type | Ingredient | CAS# | Percent |

A) Traditional Powders

| | | | |
|---|---|---|---|
| Lighting powders | | | |
| White | Titanium Dioxide | 13463-67-7 | <45 |
| | Zinc Stearate | 557-05-1 | 30 |
| | Lycopodium | 8023-70-9 | >10 |
| BiChromatic | Alumimum | 7429-90-5 | 9.5 |
| | Carbon Black | 1333-86-4 | 10 |
| Silver-Grey | Alumimun | 7429-90-5 | 19 |
| Sirchie powders: | | | |
| Hi-Fi Black Volcano, Silk | Carbon Black | 1333-8634 | ND |
| | Lycopodium | 0 | ND |
| Hi-Fi Black Volcano, Silk | Black Iron Oxide | 1317-61-9 | 36.5 |
| Carbon Free | | | |
| | Gum Arabic | 9000-01-5 | 54.5 |
| | Lycopodium | 0 | 9 |
| OPTI Black-T | Carbon Black | 1333-86-4 | 75 |
| | Talc | 14807966 | 25 |
| OPTI Black (Pumice) | Carbon Black | 1333-86-4 | 75 |
| | Pumice | 1332-09-8 | 25 |
| Hi-Fi Volcano Red | Gum Arabic | 9000-01-5 | >16 |
| | Lycopodium | 0 | >16 |
| | Pigment red 48:1 | 7585-41-3 | >65 |
| Hi-Fi Volcano Silk Grey | Aluminum | 7429-90-5 | 15 |
| | Lycopodium | 0 | 35 |
| | Titanium Dioxide | 13463677 | 40-49 |
| Hi-Fi Volcano White | Titanium Dioxide | 13463677 | 90 |
| | Zinc Stearate | 557051 | 10 |

B) Fluorescent Powders

| | | | |
|---|---|---|---|
| Lighting powders | | | |
| (their MSDS only lists | | | |
| hazardous ingredients): | | | |
| Redwop Powder | Extender | Proprietary | 80-90 |
| | Dyed Polymer | Proprietary | 10-20 |
| Greenwop Powder | Extender | Proprietary | 80 |
| | Dyed Polymer | Proprietary | 20 |

C) Metallic Powders

| | | | |
|---|---|---|---|
| Sirchie powders: | | | |
| Hi-Fi- Volcano, Silver | Aluminum | 7429-90-5 | 80 |
| | Lycopodium | 0 | 20 |
| Hi-Fi Volcano, Copper | Copper | 7440-50-8 | 14 |
| | Gum Arabic | 9000-01-5 | 58 |
| | Lycopodium | 0 | 14 |
| | Titanium Dioxide | 13463677 | 14 |

D) Fluorescent Magnetic Powders (FMP)

| | | | |
|---|---|---|---|
| Sirchie powders: | | | |
| REDcharge FMP | Iron | 7439-89-6 | 49-49.5 |
| | Lycopodium | 0 | 25 |
| | Red AX pigment | 0 | 25 |
| ORANGE charge FMP | Yellow AX Pigment | 0 | 25 |
| | Iron | 7439-89-6 | 46 |
| | Lycopodium | 0 | 25 |
| | Titanium Dioxide | 13463677 | 4 |
| GREEN charge FMP | Iron | 7439-89-6 | 46 |
| | Lycopodium | 0 | 25 |
| | Green AX Pigment | 0 | 25 |
| | Titanium Dioxide | 13463677 | 4 |
| BLUE charge FMP | Blue T Pigment | 0 | <20 |
| | Iron | 7439-89-6 | <40.5 |
| | Lycopodium | 0 | 22 |
| | Phthalocyanine blue | 147148 | >13 |
| | Titanium Oxide | 13463677 | >3.5 |
| DAZZLE Orange FMP | Yellow AX pigment | 0 | >11 |
| | Magnetite | 1317619 | <89 |
| DAZZLE Red FMP | Magneitie | 1317619 | <89 |
| | Rec AX pigment | 0 | >11 |
| DAZZLE Yellow FMP | Magnetite | 1317619 | <89 |
| | Yellow AX pigment | 0 | >11 |

Particle Sizes

Particle sizes of the fingerprint powder can vary. Typically particle sizes range from about 1 to about 50 microns. In some embodiments, about 90% of the particles are less than about 50 microns. In other embodiments, at least about 75% of the particles are less than about 30 microns. In other embodiments, at least about 50% of the particles are less than about 25 microns. In other embodiments, at least about 25% of the particles are less than about 25 microns. In other embodiments, at least about 25% of the particles are less than about 15 microns. In other embodiments, at least about 10% of the particles are less than about 25 microns. In other embodiments, at least about 75% of the particles are less than about 25 microns.

The table below lists examples of particle sizes and particle size distribution for typical print powders (in microns):

| | Powder | | | | | |
|---|---|---|---|---|---|---|
| Manufacturer | Color | <10% | <25% | <50% | <75% | <90% |
| Evident | Black | 5.7 | 11.18 | 23.62 | 26.49 | 27.89 |
| Sirchie | Black | 24.13 | 25.39 | 26.62 | 28.26 | 33.18 |
| Sirchie | Grey | NA | 10.71 | 20.61 | 27.86 | 29.68 |
| Lightning | Silver/Grey | 5.7 | 8.2 | 15.40 | 28.17 | 45.77 |
| Evident | White | 2.7 | 4.64 | 9.69 | 17.44 | 24.87 |
| Lightning | White | 1.83 | 2.37 | 3.44 | 5.65 | 9.32 |
| Lightning | RedWop | 24.73 | 26.59 | 27.72 | 28.79 | 29.65 |

Formulation Ratios

Various ratios, powder to propellant, can be used. For instance, an example of an aerosolized spray fingerprinting formulation of the present invention comprises (weight %): about 1 to about 75% fingerprinting powder; and about 25 to about 99% non-CFC propellant. In other examples, the fingerprinting powder can be present in any amount between about 1 and about 50%; between about 4 and about 30%; between about 5 and about 15%; or between about 9 and about 12%.

In other examples, the propellant can range from about 50 to about 99%; about 70 to about 96%; about 80 to about 93%; or about 85 to about 92%.

Certain embodiments of extender tubes may concentrate the sprayed formulation. In this regard, certain embodiments comprising extender tubes may have a ratio of powder to propellant in the range of 1 to 30 up to 1 to 50.

In one embodiment, the ratio is, powder to propellant, 1 to 5. In another embodiment, the ratio is 1 to 6. In another embodiment, the ratio is 1 to 7. In another embodiment, the ratio is 1 to 8. In another embodiment, the ratio is 1 to 9. In another embodiment, the ratio is 1 to 10. In another embodiment, the ratio is 1 to 11. In another embodiment, the ratio is 1 to 12. In another embodiment, the ratio is 1 to 13. In another embodiment, the ratio is 1 to 14. In another embodiment, the ratio is 1 to 15. In another embodiment, the ratio is 1 to 16. In another embodiment, the ratio is 1 to 17. In another embodiment, the ratio is 1 to 18. In another embodiment, the ratio is 1 to. In another embodiment, the ratio is 1 to 20. In another embodiment, the ratio is 1 to 21. In another embodiment, the ratio is 1 to 22. In another embodiment, the ratio is 1 to 23. In another embodiment, the ratio is 1 to 24. In another embodiment, the ratio is 1 to 25. In another embodiment, the ratio is 1 to 26. In another embodiment, the ratio is 1 to 27. In another embodiment, the ratio is 1 to 28. In another embodiment, the ratio is 1 to 29. In another embodiment, the ratio is 1 to 30. In another embodiment, the ratio is 1 to 31. In another embodiment, the ratio is 1 to 32. In another embodiment, the ratio is 1 to 33. In another embodiment, the ratio is 1 to 34. In another embodiment, the ratio is 1 to 35. In another embodiment, the ratio is 1 to 36. In another embodiment, the ratio is 1 to 37. In another embodiment, the ratio is 1 to 38. In another embodiment, the ratio is 1 to 39. In another embodiment, the ratio is 1 to 40. In another embodiment, the ratio is 1 to 41. In another embodiment, the ratio is 1 to 42. In another embodiment, the ratio is 1 to 43. In another embodiment, the ratio is 1 to 44. In another embodiment, the ratio is 1 to 45. In another embodiment, the ratio is 1 to 46. In another embodiment, the ratio is 1 to 47. In another embodiment, the ratio is 1 to 48. In another embodiment, the ratio is 1 to 49. In another embodiment, the ratio is 1 to 50.

Isolation Device

Embodiments of the present invention comprise the use of an isolation device. The isolation device is used to control and direct the misting of the aerosolized fingerprint particles.

In embodiments of the invention, the isolation device may be foldable to a collapsed position for convenient handling and storage.

The nature of the material from which the isolation device may be made is not known to be critical. In embodiments, it is made from any suitable inexpensive, durable, light-weight material, such as a polymeric material, paperboard, fabric, or the like, so that it may be discarded. For example, certain users may make an isolation device with a shirt or jacket sleeve. Of course, users may use any conceivable material, fabric, and the like that will perform as an isolation device.

The isolation device is typically conical and hollow, with the aerosol spray being discharged into the small opening and the larger opening enveloping the surface to be fingerprinted. The isolation device of the present invention does not have to form an air-tight seal over the surface, it just simply directs the mist to quickly and efficiently cover and build up the print.

The isolation device provides a chamber-like atmosphere that allows the vapors to mist and swirl around the surface to be printed. As indicated herein, the methods of the present invention allow for very little powder to be used. One advantage of this feature is that the method leaves very little space to be cleaned.

The isolation device can have a square base, round base, oval base, etc. As stated herein, the isolation device can be foldable or collapsible so that it can be easily transported, concealed, etc.

Extender Tube

Certain embodiments of the present invention comprise an extender tube. Extender tubes may decrease the amount of composition, including particles, required to identify a fingerprint and may target smaller and/or more difficult to reach areas, for example.

Embodiments of extender tubes attach to an aerosol spray container on one end and deliver the formulation through their opposite end. The tubes may be of any length or shape that meets the limitations of a particular circumstance. In certain embodiments extender tubes comprise generally elongated hollow tubes that allow for liquid communication.

Certain embodiments of extender tubes may concentrate and aim the flow of sprayed particles. Embodiments may be able to coat relatively small areas with particles, potentially with more accuracy that other methods.

Extender tubes may also be configured to reach areas that are otherwise difficult or impossible to reach with other brushed or sprayed fingerprint powder formulations.

Some embodiments of the present invention comprise extender tubes that include expanders. Expanders may be located at the end of the expander tube that is opposite from the container, or any other suitable location. Certain embodiments of expanders may otherwise be known as mechanical break up apparatuses.

Some embodiments of expanders may be designed so that particles exiting the expander tube are more evenly distributed, potentially avoiding particles from clumping together. In certain embodiments the expander is designed to receive material from the extender tube in a chamber that is connected to an exit nozzle. The chamber may be designed to swirl the material received from the extender tube, optionally through the use of rifling, walls, or other physical mean. Certain embodiments of expanders have exit nozzles that are 0.41 mm in diameter, but any diameter that sufficiently expands and disperses the composition may be implemented.

Expanders may be implemented with extender tubes that have a tendency of concentrating the sprayed composition, perhaps into a slurry. Such expanders may, in effect, re-aerosolizing the sprayed composition.

To couple an extender tube, and optionally an actuator and/or expander, to the container, the elements may be fitted together and held in place by frictional force. For example, an actuator may have a female indention in which to receive an extender tube, which is then held in place with friction. Likewise, embodiments of expanders may have a female indention to similarly receive extender tubes. It should be apparent to those of skill in the art that extender tubes, actuators, expanders, and any other element used in conjunction with embodiments of the present invention may be held in place and coupled together by any means known in the art including, but not limited to, adhesives, latches, threading the elements so they may be screwed together, and the like.

U.S. Pat. No. 6,299,674 to Takamuru et al., incorporated herein by reference, generally discusses fingerprinting methods and discloses a fingerprint detecting agent and method which can be used to detect latent fingerprints being in a wet condition.

U.S. Pat. No. 4,176,205 to Molina, incorporated herein by reference, generally discusses fingerprinting methods and discloses a fingerprint powder and a method for developing latent prints. The fingerprint powder of Molina can be applied by blowing the powder over a surface containing latent prints, or by brushing by pouring the powder on such surface to reveal a print that can be photographed or lifted by applying tape or a strippable coating over the print.

The fingerprint spray formulation of the present invention have multiple advantages in that it allows latent prints to be actualized with greater ease and with as little damage as possible to the print.

Without being bound by theory or mechanism, one advantage of the present invention when compared to traditional fingerprinting is that the present invention more evenly disperses fingerprinting powder.

Another advantage of the present invention is the use of embodiments thereof to provide a coating on a surface to indicate where there is a high probability of the location of the print. This step can be followed with any required brushing to fully expose the print for lifting. Thus, an advantage is the ability to have a controlled amount of powders to be dispensed. In certain embodiments, only one burst, for instance of a second or less, provides sufficient powder to expose and optimize the print. Therefore, the bursts limit the amount of powders present which will limit the chance a novice finger print user would overexpose powders to the surface which could over stimulate the print or even damage the print potentially beyond repair. Further, excess brushing has the potential to damage the print.

Additionally, another advantage of the present invention relates to how the composition is dispersed. The composition may be dispersed manually or in calculated bursts. In manual mode, a user may disperse composition by pressing and holding down a valve, button, and the like, thereby spraying the composition for as short or long a period as is desired. In certain embodiments, the composition may be dispersed or sprayed for calculated preset durations. Calculated present burst durations may last for about ⅓ to about 2 seconds, for example. For example, in certain embodiments a mechanical and/or electrical system may be configured such that a spray is automatically shut off after a calculated preset duration. Calculated preset burst durations allow for further refinement of bursts, increased control over the amount of composition that is dispersed, and potentially more consistent fingerprint identification.

Embodiments of the present invention may layer powder on a fingerprint incrementally. A single spray burst may form a layer satisfactory for identifying a fingerprint. Additional spray bursts can be employed to further build up powder to further expose a latent fingerprint in a predictable and user-friendly manner lessoning the chance of damaging the print. The relative ease in which embodiments of the present invention may be implemented may lower the training a forensic technician requires.

Additional embodiments of the present invention include its use in an “isolation device.” The isolation device is essentially a chamber (such as a poster tube or jacket sleeve, for example, that may be about 1 to 2 inches opening at the top and about 6 to 8 inches open at the bottom), one spray burst at the top of the tube may dispense enough powders to completely dry powder coat an object (drinking glass, knife, gun, bullet casing etc. . . . ). The tube is placed over an object or objects, one spray burst is sprayed into the top, the powder swirls inside the tube and is allowed to settle for about 5 to 10 seconds . . . then removed, and the objects are then coated ready for the final act which is the brushing to fully expose the print for lifting or photography.

Furthermore, some methods comprise spraying the fingerprint formulation on a surface, allowing the fingerprint powder to settle on to a surface, and using a brush or the like to manipulate the fingerprint powder and attempt to detect a latent fingerprint. Accordingly, since the powders need to be manipulated after they have been sprayed, in some embodiments the fingerprint formulation does not comprise any binders, adhesives, or other liquid substances, which would otherwise make the fingerprint formulation sticky and wet when sprayed.

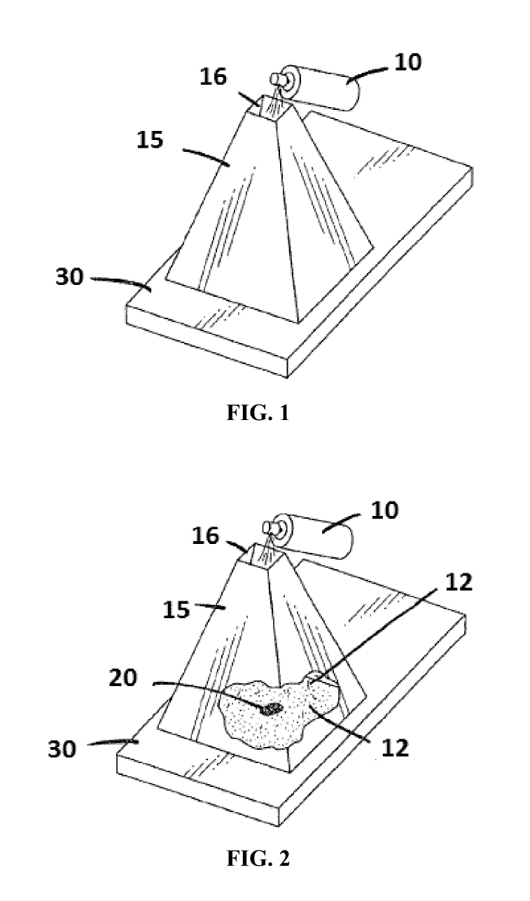

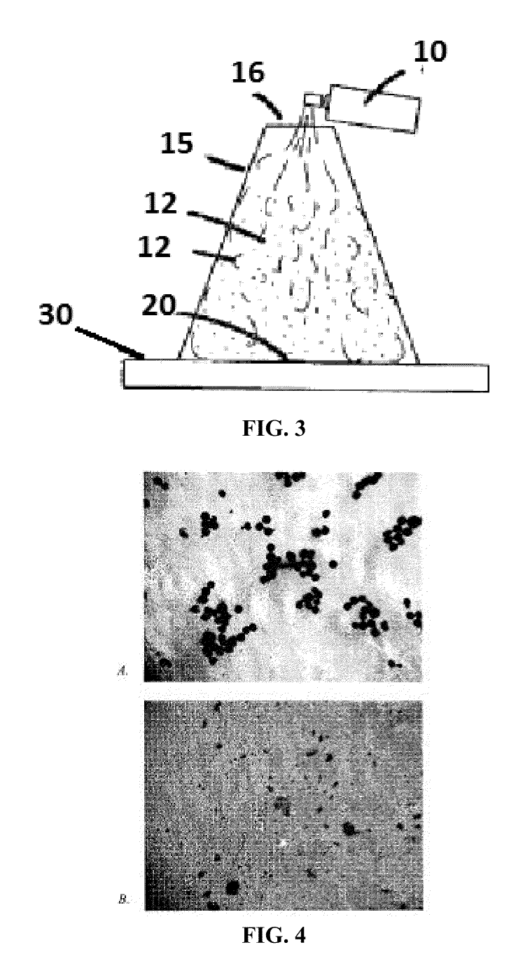

Looking to the Figures, FIGS. 1-3 show an embodiment of the present invention. The aerosolized fingerprint formulation (composition) of the present invention 10 is sprayed into an opening 16 of the isolation device 15. The isolation device is placed over a surface 30 that is suspected of or known to comprise a latent print 20. Of course, the surface can be on an object such as a glass, plate, gun, etc. The particles 12 swirl in the isolation device and help build the print.

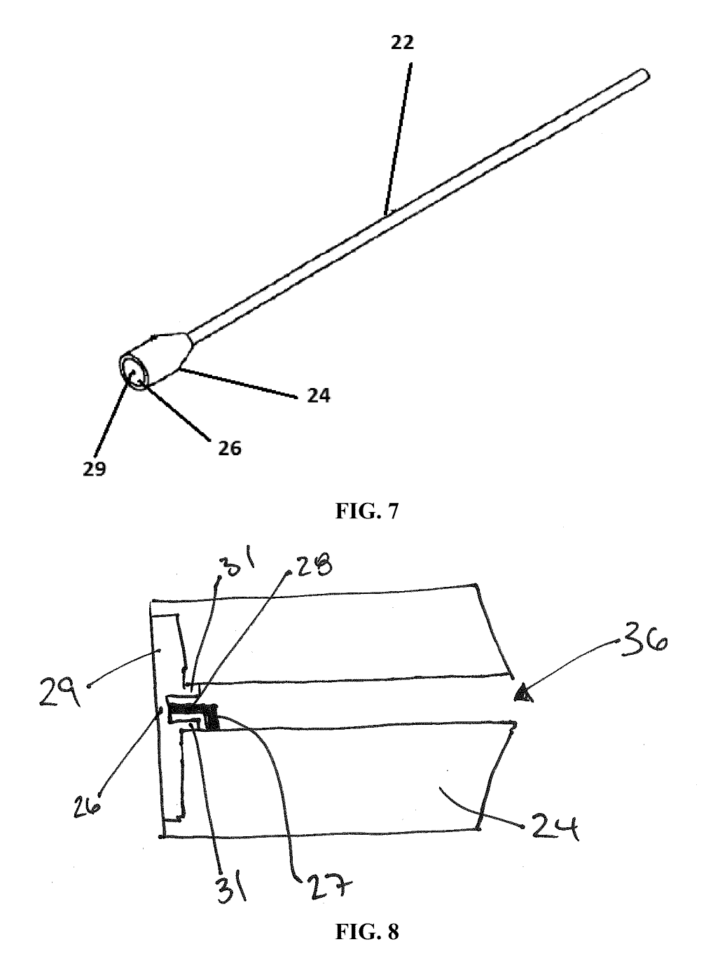

FIGS. 6 and 7 show an additional embodiment that comprises an extender tube 22 attached to the aerosolized container 10. In embodiments of the present invention, the extender tube may be used in place of the isolation device. Without being bound by theory the extender tube compresses the expelled air and particles, then an expander 24 disperses the particles on a surface. Different length tubes may be used. This embodiment allows for a small amount of particles to be required, allowing for more covert dusting of prints, if desired. Additionally, it allows for very small surfaces to be tested (such as a television switch area, for example).

FIG. 6 also depicts embodiments comprising a valve stem 25 that may protrude outwardly from the aerosolized container 10. An actuator 23 may be coupled to the valve stem 25 and/or the extender tube 22. In certain embodiments the actuator 23 is permanently attached to the extender tube 22. In certain embodiments the extender tube may be directly coupled to the valve stem 25. In further embodiments, a user may detach and reassemble the aerosolized container 10, actuator 23, and extender tube 22.

Looking now to FIGS. 8 to 10, a specific embodiment of an extender tube 22 and expander 24 are shown. Like the previously described embodiments, the embodiment comprises an extender tube 22 that can be frictionally connected to an expander 24. The expander 24 can be coupled to an extender tube 22 on one end side and can comprise an exit cap 29 coupled to its other end side that comprises an exit nozzle 26. Accordingly, a fingerprint formulation can enter one end of the extender tube 22, pass through the extender tube 22, enter into the expander 24, exit through the exit nozzle 26 located on the exit cap 29, and deposit onto a surface 30 that potentially has a latent print thereon.

As shown in FIG. 8, the expander 24 is configured to manipulate a pressurized stream or burst of fingerprint formulation so that the fingerprint formulation will be released via the exit nozzle 26 as a fine mist, rather than as a stringy and/or concentrated stream. The embodied expander 24 comprises an expander inlet 36 through which the fingerprint formulation can first enter into the expander 24.

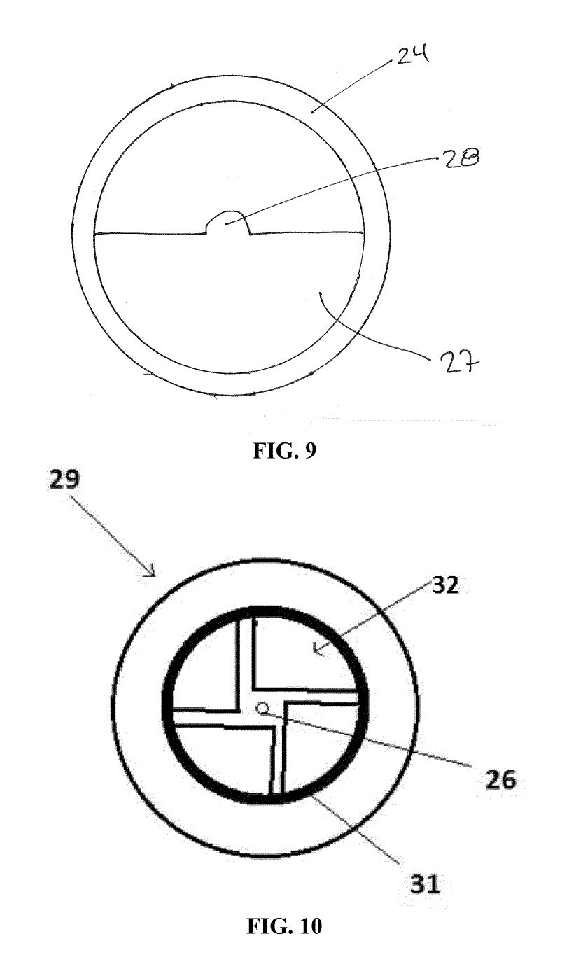

After the fingerprint formulation enters the expander 24, it reaches a divider 27 that separates an exit chamber 34 of the expander 24 from a side of the expander 24 located towards the inlet 36. More particularly, as shown in FIG. 9, the divider 27 can have a semicircular shape and can block the flow of fingerprint formulation at least with respect to a portion of the inner area of the expander 24. In the embodied expander 24, the divider 27 blocks about 50% of the area inside the expander 24, but in other embodiments the divider can block about 20%, about 40%, about 60%, or about 80% of the area of the expander 24.

Looking still to FIG. 8, the expander 24 comprises a projection 28 that extends from the divider 27 toward the exit nozzle 26. The projection 28, when viewing the expander 24 from a longitudinal cross section, occupies the center of the expander 24. In this manner, the projection 28 has a void that surrounds it and that defines a chamber 34 located on the exit nozzle 26 side of the divider 27. In the particular embodiment, the projection 28 extends so that it is in contact or nearly in contact with an inner surface of the exit cap 29.

In this regard, the exit cap 29 defines an end of the expander 24 through which fingerprint formulation may be sprayed. The exit cap 29 comprises an exit nozzle 26 at center portion thereof. The exit cap 29 further comprises a tubular projection 31 that can extend into the expander 24 and can be dimensioned so that an exterior surface of the tubular projection 31 is frictionally engaged by an interior surface of the expander 24. The tubular projection 31 of the exit cap 29 can be dimensioned so that the chamber 34 is defined by a void located between the tubular projection 31 and the outer surface of the projection 28. Further still, in some embodiments the tubular projection 31 of the exit cap 29 can extend so that it is in contact with the divider 27.

Looking now to FIG. 10, a view of the exit cap 29, as seen from an interior of the expander 24, is shown. At the center of the exit cap 29 there can be an exit nozzle 26 through which fingerprint formulation can exit. An area on the surface of the exit cap 29 located between the exit nozzle 26 and the tubular projection 31 can comprise a patterning 32. In the depicted embodiment, the patterning 32 comprises four raised corners that converge toward the exit nozzle 26 and that are offset relative to one another. Consequently, the patterning 32 comprises four recessed areas that are defined by the raised corners and that generally extend from the tubular projection 31 toward the exit nozzle 26, and preferably toward an edge of the exit nozzle 26. When fully assembled, the projection 27 can be adjacent to or in close proximity to the raised portions of the patterning 32.

In this respect, the embodiment depicted in FIGS. 8 to 10 is configured to impart spray characteristics that can be desirable for certain fingerprint detection situations. In particular, a fingerprint formulation can exit an aerosolized container 10 via the valve stem 25, pass through the extender tube 22, enter the expander 24, and exit the expander 24 via the exit nozzle 26 as a finely dispersed spray. Within the expander 24, the fingerprint formulation can be propelled towards the exit cap 29, and once the fingerprint formulation contacts the exit cap 29 the patterning 32 can further manipulate the flow of the fingerprint formulation. For instance, if the projection 27 contacts or is near the patterning 32, the fingerprint formulation will generally move from an outer edge of the patterning 32 towards the exit nozzle 26 located at the center of the exit cap 29 along the recessed portions formed in the patterning 32. Since the patterning 32 is offset, the patterning 32 can impart a swirling characteristic to the fingerprint formulation prior to it exiting via the exit nozzle 26.

Accordingly, the specific embodiment of an expander 24 depicted in FIGS. 8 to 10 can be configured to manipulate the flow of a fingerprint formulation so that it dispenses as a fine mist. On the other hand, in some embodiments a spray that exits from an extender tube 22 having an approximately uniform tubular cross-section can produce a stream of fingerprint formulation that relatively stringy and/or concentrated. The described embodiments is, of course, only intended to illustrate one possible embodiment, and those of ordinary skill will appreciate modifications or variations to the described embodiment that can be made without departing from the scope of the presently-disclosed subject matter.

Embodiments of isolation devices and extender tubes may be used together or independently of one another.

### EXAMPLES

The following examples are presented to further illustrate the invention. But, it should be recognized that the invention is not to be considered limited thereto.

### Example 1

This Example is a preferred fingerprint composition. About 2 grams of Lightning White™ fingerprint powder is placed in an aerosol container manufactured by CCL Container Corporation, Hermitage, Pa. The container is provided with two steel mixing balls that are about 4 to 6 mm in diameter. A dip tube is asserted in the container and then the container is crimped and sealed. An aerosol valve is supplied by Summit Packaging Systems, Inc., Manchester, N.H. In this example, the valve assembly comprises an actuator (Part. No. B-77/97), stem (77148), stem gasket (77505), spring (77401), body (97311), dip tube (200610), and mounting cup (77792). The crimped aerosol container is then charged with a hydrocarbon butane propellant to a pressure of about 31 psig at 70 degrees Fahrenheit. The product is actualized with 20 grams of A-31 propellant. The final actuator button is fixed and the product is ready for spraying.

### Example 2

This Example demonstrates four exemplary embodiments of the present invention. These non-limiting example incorporate four different aerosol powders using various container sizes filled with particular powder masses (small container: 1 g powder, medium container: 4 g powder, large container: 7 g powder). Two different non-porous substrates bearing latent print impressions from an oil standard of varying deposition intensity and ages were used in this example. Half of the impressions were processed using cyanoacrylate ester (CNA) prior to the application of the powder. The other half of the impressions were processed directly with the aerosol powder fingerprint formulations of the present invention. Results indicate this process to be an effective technique on non-porous surfaces without CNA. In summary, this example shows that the present invention is an effective fingerprinting method, and that it maintains a relatively even distribution of powder, controls the amount of powder deposited, and decreases most of the brush contact with the surface thereby lessening the chances of damage to impressions

Materials and Methods: Four commercial fingerprint powders of the most readily used brands (Sirchie, Lightning, Peavey, Tri-Tech, and Evident) are prepared in aerosol containers of various sizes (small, medium, and large) and filled with particular powder masses, 1 g, 4 g, and 7 g, respectively, maintaining a similar powder to propellant (about 10 to about 1)

The environment for this example comprised a temperature and humidity of about 72.5 degrees Fahrenheit and 21%, respectively.

A commercial sebaceous control matrix standard (Armor Forensics #1-2792) was used for the deposition of the latent print impressions on two types of non-porous substrates—smooth and textured plastic. Thirty-six latent print impressions were deposited on each type of substrate within each particular deposition age—zero days, seven days, and fourteen days. The depositions consisted of three successive latent print impressions decreasing in deposition intensity (amount of matrix available for deposition) for a total of twelve gradients. An additional twelve gradients were processed with CNA prior to the application of powders. Each of the twenty-four gradients (twelve gradients processed with CNA and twelve gradients without) were subject to development by each of the four particular powders distinguished by the three aerosol container sizes containing a particular powder mass per container.

The application of the aerosolized powders for each of the latent print impressions was as follows: (a) the isolation device, a small cone with openings at each end, was placed over the area of interest containing the impression to retain the powder in the desired area of application; (b) each aerosol container was thoroughly shaken for 10-15 seconds to maintain the powder consistency and spray distribution; (c) each aerosol container was directed into the isolation device and a single burst was sprayed for approximately ⅓ second up to about 1 second; (d) the powder was allowed to settle for approximately 10 seconds prior to removing the isolation device; and (f) the area of interest was lightly dusted with a fiberglass brush (Evident #1008) to remove excess powder deposition.

All latent print impressions developed were digitally captured (Nikon D2Xs) prior to a visual analysis of the printed images (Xerox Phaser 7750DN PS) by nine certified latent print examiners. Under single blind procedures each certified latent print examiner judged the developmental quality of each of the 432 latent print impressions according to the clarity of the developed impression by assigning a numerical rating on a scale from zero to five based on the following: 0=No development; 1=Poor development; ridge structure unclear; 2=First level detail—visible pattern type with unclear ridge path configurations; 3=Second level detail present—visible ridge path configurations; 4=Good development—clear and distinct ridge path configurations; 5=Excellent development—clear ridge path configurations with distinct ridge and pore structure.

Additionally, each examiner was provided with the following definition for clarity: “Clearness, i.e., how well friction skin detail is recorded in a print. In other words, how well the details from 3-D ridges are reproduced in the 2-D print is referred to as the clarity of the print. When most of the detail found on the friction ridges is reproduced in the friction ridge print, the print is considered clear. If few of the details from the friction ridges are reproduced, the print is considered unclear.” See Ashbaugh, D. Quantitative-Qualitative Friction Ridge Analysis. CRC Press. 1999.

All images were presented to each examiner in a format so that only the study coordinators were aware of the image identity. Data was recorded and plotted in a format similar to the answer sheets provided to each examiner for interpretation.

Results: The ratings given by the examiners for each image were averaged thereby revealing an overall rating for each image that can be related back to a level of development listed on the rating scale. These averaged ratings were used to compare substrate types, powder colors, prior processing, deposition intensity, container size, and age. Impressions receiving an average rating of 3.0 or higher are considered to be suitable for comparative purposes.

### Substrate Type

An overall comparison of the developmental quality of the impressions on smooth versus textured surfaces for each specific age of impression is illustrated in Table 1. These results indicate no significant difference in the overall rating for those impressions developed on either substrate for each age. It was noted, however, that Day zero impressions on both surfaces were equivalent in comparative value whereas Days seven and fourteen, while similar in their overall rating, were not suitable for comparative purposes.

TABLE 1
An overall comparison of the developmental quality of the impressions
on smooth versus textured surfaces for each specific age.

| | Smooth | Textured |
|---|---|---|
| Day 0 | 3.3 | 3.4 |
| Day 7 | 2.8 | 2.5 |
| Day 14 | 2.6 | 2.2 |

### Powder Colors

Table 2 concentrates on the ratings of the individual powders used and the effect of age on their developmental capabilities. Based on this information, it can be quickly gathered that the black powder was less effective in all ages when compared to the grey, white, and fluorescent powders. The highest quality of development came from both the grey and white powders presenting relatively equal quality ratings for all ages—both of which were suitable for comparative purposes for all ages, except Day fourteen, although close in proximity did not meet the threshold. The fluorescent powder followed in developmental quality behind white and grey powders, while still meeting the threshold but only on Day zero, Day seven impressions fell just under the threshold while Day fourteen impressions were not suitable for comparison.

TABLE 2
An overall comparison of the developmental quality of the impressions
developed with various powders for each specific age.

| | Grey | White | Black | Fluorescent |
|---|---|---|---|---|
| Day 0 | 4.0 | 4.2 | 2.1 | 3.1 |
| Day 7 | 3.2 | 3.4 | 1.3 | 2.8 |
| Day 14 | 2.9 | 2.9 | 1.3 | 2.5 |

### Prior Processing

With relation to the impressions processed with CNA and those that were not, Table 3 demonstrates a comparison of the overall ratings between those impressions. More specifically, impressions not processed with CNA prior to powder application maintained a more consistent developmental quality throughout all ages, whereas those that were processed with CNA exhibited a considerable drop in quality past Day zero resulting in development too poor for comparative purposes.

TABLE 3
An overall comparison of the developmental quality of those impressions
previously processed with CNA versus those that were not.

| | CNA | Non-CNA |
|---|---|---|
| Day 0 | 3.0 | 3.6 |
| Day 7 | 1.9 | 3.4 |
| Day 14 | 1.6 | 3.2 |

### Deposition Intensity

For each of the three successive deposition intensities per age, Table 4 breaks down the combined ratings for each deposition. Overall, the development of each deposition intensity differed very little between ages, suggesting age had little to no effect on developmental capabilities. Deposition one impressions were superior in quality by meeting the threshold for comparative purposes; however, depositions two and three impressions did not develop to a comparable degree across all ages.

TABLE 4
An overall comparison of the developmental quality on successive
deposition intensities (amount of matrix available for development).

| | Deposition 1 | Deposition 2 | Deposition 3 |
|---|---|---|---|
| Day 0 | 3.5 | 2.7 | 2.5 |
| Day 7 | 3.4 | 2.8 | 2.4 |
| Day 14 | 3.1 | 2.6 | 2.2 |

### Container Size

A comparison of the quality of the impressions developed using various container sizes filled with particular powder masses (small container: 1 g powder, medium container: 4 g powder, and large container: 7 g powder) for each particular powder is illustrated in Tables 5 through 7 distinguished by age. These results indicate that for the majority of the cases difference in the container sizes and powder masses did not affect the quality of development. Interestingly for fluorescent powder, however, for both Days seven and fourteen, there was a noticeable difference in the developmental quality between the small container and the large container to such a degree that only those impressions developed with the large container were suitable for comparative purposes.

TABLE 5
An overall comparison of the developmental quality of the impressions
on Day zero using various container sizes for each particular powder.

| | Small Container | Medium Container | Large Container |
|---|---|---|---|
| | (1 g powder) | (4 g Powder) | (7 g Powder) |
| Grey | 3.9 | 4.2 | 3.9 |
| White | 4.3 | 4.1 | 4.1 |
| Black | 2.1 | 2.0 | 2.0 |
| Fluorescent | 3.0 | 3.2 | 3.1 |

TABLE 6
An overall comparison of the developmental quality of the impressions
on Day seven using various container sizes for each particular powder.

| | Small Container | Medium Container | Large Container |
|---|---|---|---|
| | (1 g powder) | (4 g Powder) | (7 g Powder) |
| Grey | 3.0 | 3.1 | 3.4 |
| White | 3.2 | 3.3 | 3.5 |
| Black | 1.4 | 1.2 | 1.5 |
| Fluorescent | 2.3 | 2.9 | 3.2 |

TABLE 7
An overall comparison of the developmental quality of the impressions on
Day fourteen using various container sizes for each particular powder.

| | Small Container | Medium Container | Large Container |
|---|---|---|---|
| | (1 g powder) | (4 g Powder) | (7 g Powder) |
| Grey | 2.8 | 2.9 | 3.1 |
| White | 2.5 | 3.2 | 3.0 |
| Black | 1.0 | 1.1 | 1.6 |
| Fluorescent | 1.7 | 2.5 | 3.4 |

### Deposition Age

A comparison of the quality of the impressions developed at different ages indicates decreasing developmental quality as the age of the impression increases. Overall, Day zero impressions were suitable for comparative purposes having an average rating of 3.3, whereas Days seven and fourteen impressions were not, having a rating of 2.7 and 2.4, respectively.

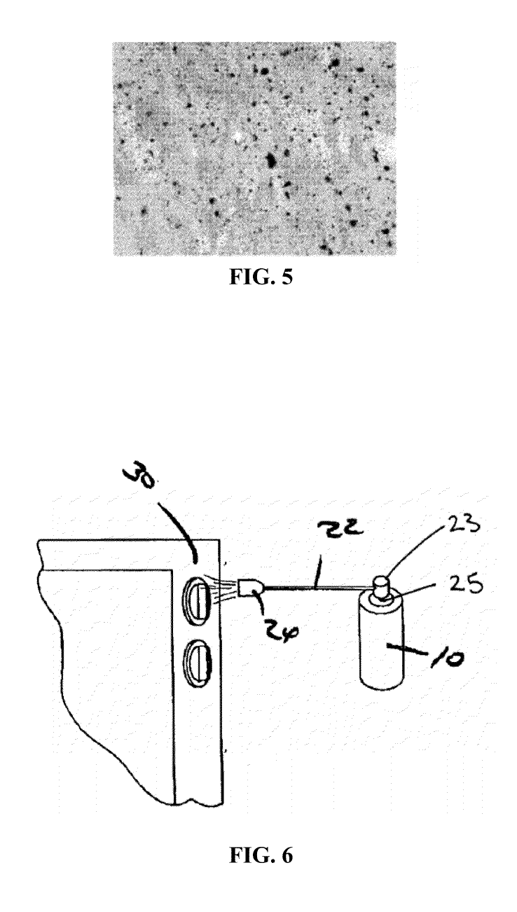

In relation to the developmental capabilities of the different powders used in this study, it is interesting to note the difference in the quality of the impressions developed with the black powder as compared to the white and grey powders. This may be attributable to the brands of powders used or the powder composition and morphology of the individual particles which is illustrated in FIGS. 4 and 5. In addition to the observations of the black powder, the fluorescent powder exhibited moderate development overall that may be explained, in part, because the powder had a tendency to create speckling in and around the furrows of the impression which may have contributed to the inferior quality observed in relation to the white and grey powders.

The effect of CNA processing prior to the application of the powders resulted in little to no development at times, whereas those impressions of the same age with no prior processing maintained discernible ridge detail. These results may be related to the age of the impressions having a possible negative affect on CNA development, which then, may have affected the powders' ability to develop those impressions. Based on this observation, it appears that this method would not be conducive for use in combination with CNA. Further studies are warranted as to why these results were observed.

Multiple deposition intensities were evaluated to ascertain the sensitivity of the powders to develop quality impressions. The quality of development declined for each successive deposition intensity culminating with deposition three (least amount of matrix available for development) maintaining a moderate level of quality development by having an average rating of 2.4 for all ages combined.

Although three separate powder masses were tested, each from a different container size, these variables did not contribute to a significant difference in the developmental quality, probably due to the fact that all containers contained a similar powder to propellant ratio. However, the differences in the developmental quality of fluorescent powder, as noted in Tables 6 and 7, between the small and large containers are interesting, but warrant further studies with particular emphasis on the possible factors which may have contributed to this result.

While the age of the impression did seem to have an affect on the powders' ability to develop the impressions, Day fourteen impressions were still developed with a moderate level of quality having an average rating of 2.4. From this observation it would appear this method would not be precluded from use on impressions exposed for a couple of weeks; however, it should be cautioned that normal environmental conditions may differ to a considerable degree when compared to the controlled laboratory settings of this study.

As shown herein, the methods of the present invention are a simple and convenient process that was very effective in controlling the amount of powder deposited on the substrate surface allowing for the powder to be evenly distributed. Additionally, the present invention is a significant improvement over the prior art.

When the initial spray did not immediately develop the impressions, the powders did reveal an outline of the impressions which could then be fully developed after a light brushing using a clean fiberglass fingerprint brush each time. By using the isolation device, the powder was effectively contained while allowing it to settle around the area of interest, which also cuts down on health risks of airborne powders, cleanup, etc. Additionally, the present invention is an effective and less challenging technique, especially for the inexperienced. It helped by maintaining a relatively even distribution of spray, controlling the amount of powder deposited, and decreasing brush contact with the surface thereby lessening the chances of damage to the impression.

### Example 3

This Example is a preferred fingerprint composition for certain embodiments comprising an extender tube. About 0.5 grams of Lightning White™ fingerprint powder is placed in an aerosol container manufactured by CCL Container Corporation, Hermitage, Pa. The container is provided with two steel mixing balls that are about 4 to 6 mm in diameter. A dip tube is asserted in the container and then the container is crimped and sealed. An aerosol valve is supplied by Summit Packaging Systems, Inc., Manchester, N.H. In this example, the valve assembly comprises an actuator (Part. No. B-77/97), stem (77148), stem gasket (77505), spring (77401), body (97311), dip tube (200610), and mounting cup (77792). The crimped aerosol container is then charged with a hydrocarbon butane propellant to a pressure of about 31 psig at 70 degrees Fahrenheit. The product is actualized with approximately 20 grams of A-31 propellant. The final actuator button is fixed and the product and an extender tube is fitted into the opening provided on the actuator. The product is then ready for spraying.

### Example 4

This Example is describes methods for utilizing embodiments comprising an extender tube and the advantages that can be obtained therefrom. A user may suspect or come to learn that a latent print is located on a certain object or surface. A user will then obtain a aerosolized container containing, for example, the composition described in Example 3. In the event that an extender tube is not already coupled to the container, the user will have to do so. In certain embodiments the extender tube is directly coupled to the container, for example, onto a valve stem of a container. In other embodiments the extender tube will comprise an actuator, and the actuator will be coupled to the container. In further embodiments the actuator is already coupled to the container, and the extender tube may be attached to the actuator. The actuator may or may not be a permanent element of either the extender tube or the container. One of ordinary skill in the art may appreciate that any feasible combination of containers, actuators, extender tubes, and the like may be used to couple the extender tube to the container without departing from the spirit of the present invention.

A user will then point the end of the extender tube toward the suspected or known latent print and spray the compositions. The composition may come out for as long as the user activates the container. In other embodiments, the composition will come out in preset calculated bursts. For example, a user may activate the container and a 0.5 second burst of composition will exit from the end of the extender tube, projecting the composition onto the latent print. Multiple such bursts may be applied if necessary, either manually or via calculated preset bursts.

The extender tube may be used to focus a burst on a small object or a small area, such as a keyboard, shell casing, and the like. The extender tube may also target hard to reach areas, such as those behind large objects or in tight crevices, for example. The extender tube may also be modified in length or shape to reach and/or reach around certain objects.

A user may further implement an isolation device in conjunction with an extender tube. In such embodiments, a user may reap the benefits provided by both the isolation device and the extender tube.

In certain embodiments an expander is provided with the extender tube. In such embodiments, the expander will redistribute the composition exiting from an extender tube, thus ensuring that the spray is kept uniform and that the composition does not clump to the extent that it may otherwise.

Following this procedure, a user may subsequently use methods known in the art and those described herein to identify and analyze a latent print.

As described above, numerous benefits will result from employing the concepts of the present invention composition.

The invention thus being described, it would be obvious that the same may be varied in many ways. Such variations should not be regarded as a departure from the spirit and scope of the present invention, and all such variations as would be obvious to one of ordinary skill in the art are intended to be included within the scope of this invention.

This application references various patents and/or publications. All such patents and/or publications are expressly incorporated herein by reference in their entirely.

Finally, unless otherwise indicated, all numbers expressing quantities of ingredients, reaction conditions, and so forth as used in the specification and claims are to be understood as being modified by the term “about.” Accordingly, unless specifically indicated to the contrary, the numerical parameters set forth in this specification and claims are approximations that may vary depending on the desired properties sought to be obtained by the present invention.

Notwithstanding that the numerical ranges and parameters setting forth the broad scope of the inventions are approximations, numerical values set forth in the specific examples are reported as precisely as possible.

We claim:

1. A method of obtaining fingerprints, comprising:
providing an aerosolized container containing a composition, wherein the composition includes at least one fingerprint powder and a dry propellant;
coupling an extender tube to the container;
identifying a surface that may contain a latent fingerprint;
aiming the extender tube at the surface; and
spraying the composition through the extender tube onto the surface.

2. The method of claim 1, further comprising:
lifting the latent fingerprint.

3. The method of claim 1, further comprising:
analyzing the latent fingerprint.

4. The method of claim 1, wherein the composition comprises a ratio of from about 1 to 26, powder to propellant, to about 1 to 50, powder to propellant.

5. The method of claim 1, wherein the composition comprises a ratio of about 1 to 40, powder to propellant.

6. The method of claim 1, wherein an expander is coupled to an end of the extender tube that is opposite an end of the extender tube that is coupled to the container.

7. The method of claim 1, wherein the composition further comprises a second fingerprint powder.

8. The method of claim 1, wherein the spraying step comprises spraying one or more bursts of the composition, and wherein each of the one or more burst lasts for a burst duration.

9. The method of claim 8, wherein the burst duration is about ⅓ seconds to about 2 seconds.

10. The method of claim 8, wherein the burst duration is a calculated preset duration.

11. A kit, comprising:
an aerosolized container containing a composition, wherein the composition includes a fingerprint powder and a dry propellant; and
an extender tube, wherein the extender tube includes a first end and a second end.

12. The kit of claim 11, wherein the first end of the extender tube may be coupled to the container such that the extender tube is in fluid communication with the composition.

13. The kit of claim 11, further comprising:
an expander, wherein the expander is configured to disperse the composition.

14. The kit of claim 3, wherein the expander is coupled to the second end of the extender tube.

15. The kit of claim 11, wherein the propellant is a non-flammable propellant.

16. The kit of claim 15, wherein the non-flammable propellant is a hydrofluorocarbon.

17. The kit of claim 16, wherein the hydrofluorocarbon is selected from the group consisting of 1,1,1,2-tetrafluoroethane, 1,1,1,3,3,3-hexafluoropropane, 1,1,1,2,3,3,3-heptafluoropropane, and combinations thereof.

18. The kit of claim 1, further comprising:
a second fingerprint powder.

19. The kit of claim 18, wherein the fingerprint powder and second fingerprint powder are selected from the group consisting of traditional powders, fluorescent powders, metallic powders, and fluorescent magnetic powders.

20. The kit of claim 18, wherein the fingerprint powder is a traditional black powder, and wherein the second fingerprint powder is an orange fluorescent powder.

21. The kit of claim 11, further comprising at least one of a second container containing the composition, gloves, an ultraviolet light, a flashlight, a camera, lifting tape, and a brush.

22. An aerosolized spray apparatus comprising:
a container containing a composition, wherein the composition includes a fingerprint powder and a dry propellant; and
an extender tube, wherein the extender tube includes a first end and a second end, and wherein the first end is coupled to the container such that the extender tube is in fluid communication with the composition.

23. The aerosolized spray apparatus of claim 23, further comprising an expander coupled to the second end of the extender tube.
